(19) United States
(12) Patent Application Publication
(10) Pub. No.: US 2023/0413161 A1
(43) Pub. Date: Dec. 21, 2023

(54) PUSH NOTIFICATION FOR MEDICAL DEVICES

(71) Applicant: Boston Scientific Neuromodulation Corporation, Valencia, CA (US)

(72) Inventors: Adarsh Jayakumar, Valencia, CA (US); Joshua Uyeda, Los Angeles, CA (US); Lisa Denise Moore, Glendale, CA (US); Andrew James Haddock, Los Angeles, CA (US)

(21) Appl. No.: 18/195,172

(22) Filed: May 9, 2023

Related U.S. Application Data
(60) Provisional application No. 63/344,701, filed on May 23, 2022.

(51) Int. Cl. H04W 48/10; H04W 48/16; H04L 5/00

(52) U.S. Cl. CPC H04W 48/10; H04L 5/0048; H04W 48/16

(57) ABSTRACT

A system may include a first device and a second device. The first device may be configured to send advertising packets with a request to communicate with the second device, and the second device may be configured to discover the first device and initiate a wireless communication session with the first device using the advertising packets. The first device may be configured to send advertising packets and send a request to communicate with the second device, and the second device may be configured to discover the first device and initiate a wireless communication session with the first device using the advertising packets.

### CLAIM OF PRIORITY

This application claims the benefit of U.S. Provisional Application No. 63/344,701, filed on May 23, 2022, which is hereby incorporated by reference in its entirety.

### TECHNICAL FIELD

This document relates generally to medical systems, and more particularly, but not by way of limitation, to systems, devices, and methods that provide push notification for a medical device.

### BACKGROUND

Medical devices may include therapy-delivery devices configured to deliver a therapy to a patient and/or or monitors configured to monitor a patient condition via user input and/or sensor(s). For example, therapy-delivery devices for ambulatory patients may include wearable devices and implantable devices, and further may include, but are not limited to, stimulators (such as electrical, thermal, or mechanical stimulators) and drug delivery devices (such as an insulin pump). An example of a wearable device includes, but is not limited to, transcutaneous electrical neural stimulators (TENS), such as may be attached to glasses, an article of clothing, or a patch configured to be adhered to skin. Implantable stimulation devices may deliver electrical stimuli to treat various biological disorders, such as pacemakers to treat cardiac arrhythmia, defibrillators to treat cardiac fibrillation, heart failure cardiac resynchronization therapy devices, cochlear stimulators to treat deafness, retinal stimulators to treat blindness, muscle stimulators to produce coordinated limb movement, spinal cord stimulators (SCS) to treat chronic pain, cortical and Deep Brain Stimulators (DBS) to treat motor and psychological disorders, Peripheral Nerve Stimulation (PNS), Functional Electrical Stimulation (FES), and other neural stimulators to treat urinary incontinence, sleep apnea, shoulder subluxation, etc.

The medical device, such as an implantable or wearable device, may sense or otherwise generate data, and it may be desirable to communicate that data to a controller or other device. However, various wireless communication protocols, such as Bluetooth Low Energy (BLE), do not accommodate “push notification” functionality for the peripheral role devices (e.g., stimulator) to communicate critical data (e.g., emergency data) to the external controllers in a secure and private manner. It is noted that the BLE does allow for “push-like notifications” for broadcast role devices, but this data can be seen by any nearby scanning device. However, when a peripheral and external are previously paired and bonded, the peripheral role device has not been able to alert the external device, functioning as a central role device, that the peripheral role device needs to communicate with it in a secure and private manner. The wireless communication may manage the protocols used to initiate wireless communication between device. Some other devices may advertise their presence and others may control whether to initiate the communication. For example, the GAP (Generic Access Profile) defines the discovery, connection and link management part of Bluetooth communication. A GAP peripheral device broadcasts information about itself using advertisements, and a GAP central device scans for advertisements and analyzes the advertisements found in the scan result. The central device can decide to connect to an advertising peripheral. Peripheral devices may send BLE advertising packets out on 3 channels—37 (2402 MHz), 38 (2426 MHz), 39 (2480 MHz) for each advertising event at an advertising interval which specifies the time between advertising events, and then may for connection requests on the channel the advertising packet was just sent out on for a specified amount of time. The connection request sent from the central device to the peripheral device may include connection parameters for the new connection. The parameters may include a connection interval indicating how often the peripheral and central devices communicate and a channel map identifying the channels on which they will communicate. The peripheral may accept the connection by tuning into the right frequency at the right time to establish the connection. The central and peripheral devices continue to communicate at the identified channels and times as long as the connection is established.

Medical device communications may use BLE to communicate because of its benefits such as low power and because it is relatively inexpensive. Further, BLE is an industry standard that is supported by consumer devices such as phones and laptops. For example, medical devices, such as implantable medical devices like implantable neurostimulators, may be configured as GAP peripheral role devices while the external controllers, such as remote controls (RCs), clinician programmers (CPs), phones, and the like may be configured as GAP central role devices that must initiate a BLE connection (i.e., scanning, connecting, pairing, bonding, and app-level security). Alternatively, external devices may be configured as GAP peripheral role devices and the medical device may be configured as a GAP central role device. The BLE communication design may not allow application data to be transmitted outside of a BLE connection. Since a connection consumes more power than advertising, a BLE connection model may not maintain long running connections between the controller and the medical device (e.g., an implantable medical device (IMD) such as an implantable neurostimulator). Therefore, there is no mechanism for the IMD to initiate the communication of application data in the default (unconnected) BLE state. Further, it is undesirable to embed application data into the BLE advertising packet that is transmitted by the GAP peripheral because of privacy concerns related to the data being exposed and the ability of other BLE scanning devices to track the IMD.

What is needed is the ability for a medical device to produce a push notification to accommodate emergencies when it is not authorized to initiate a communication session with another device. For BLE communication, what is needed is for a medical device configured to function as a GAP peripheral device to produce a push notification to a GAP central device.

### SUMMARY

An example (e.g., “Example 1”) of a system may include a system that includes a first device and a second device. The first device may be configured to send advertising packets with a request to communicate with the second device, and the second device may be configured to discover the first device and initiate a wireless communication session with the first device using the advertising packets.

In Example 2, the subject matter of Example 1 may optionally be configured such that the first device includes a medical device configured for use in sensing at least one health-related condition, delivering a therapy, or both sensing the at least one health-related condition and delivering the therapy.

In Example 3, the subject matter of Example 2 may optionally be configured such that the medical device includes an implantable medical device.

In Example 4, the subject matter of any one or more of Examples 1-3 may optionally be configured such that the first device includes a neuromodulator configured to deliver a neuromodulation therapy or a cardiac stimulator configured to deliver electrical therapy to a heart.

In Example 5, the subject matter of any one or more of Examples 1-4 may optionally be configured such that the first device is configured to determine a trigger for requesting communicating with the second device, and respond to the determined trigger by sending the request. The first device may request the second device to initiate communication by including embedded data in the advertising packets, and the embedded data may signal a request by the first device for the second device to initiate a communication connection. The subject matter may be configured such that the second device is configured to scan for advertising packets to discover the first device, recognize the embedded data in the advertising packets, and initiate the communication connection with first device in response to recognizing the embedded data. By way of example and not limitation, the first device may be configured as a BLE GAP peripheral device and the second device may be configured as a BLE GAP central device.

In Example 6, the subject matter of Example 5 may optionally be configured such that the first device is configured to send the advertising packets according to an advertising interval that specifies time between advertising events, and modify the advertising interval in response to the determined trigger.

In Example 7, the subject matter of Example 6 may optionally be configured such that the first device is configured to determine more than one condition, the method further comprising using the first device to determine at least one condition from the more than one condition, and selecting the advertising interval based on the determined at least one condition, wherein different conditions correspond to different advertising intervals.

In Example 8, the subject matter of any one or more of Examples 5-7 may optionally be configured such that the embedded data in the advertising packets is a bit to signal the request by the first device for the second device to initiate the communication connection.

In Example 9, the subject matter of any one or more of Examples 5-7 may optionally be configured such that the first device is configured to determine more than one condition, the determined trigger includes at least one condition from the more than one condition, and the first device is configured to identify the determined at least one condition in the embedded data within the advertising packets.

In Example 10, the subject matter of Example 9 may optionally be configured such that the first device is configured to send a time stamp within the embedded data corresponding to when the at least one condition occurred.

In Example 11, the subject matter of any one or more of Examples 9-10 may optionally be configured such that the first device is configured to send additional information relevant to the determined at least one condition, and the additional relevant information includes: an action by the first device, at least one measurement performed by the first device, or an indication of a condition of the first device or a person using the first device.

In Example 12, the subject matter of any one or more of Examples 5-11 may optionally be configured such that the first device is configured to change operation based on the determined trigger.

In Example 13, the subject matter of Example 12 may optionally be configured such that the first device changes between a closed-loop therapy and an open loop therapy based on the determined trigger.

In Example 14, the subject matter of any one or more of Examples 5-11 may optionally be configured such the determined trigger includes a determined medical event or a determined device event for the first device. Examples of medical events may include, but are not limited to, accelerometer-detected signals that may be used to determine falls, heart rate variability, and respiration, and electrical signals sensed through a lead such as heart rate and neural activity. Examples of device events that may be determined by the first device may include, but are not limited to, changes in therapy (e.g., stimulation unexpectedly off), battery is low and is need of recharge, battery is near end of life, the device has changed modes of operation (e.g., a fallback mode such as open loop therapy rather than closed loop therapy).

In Example 15, the subject matter of any one or more of Examples 1-4 may optionally be configured such that at least one of the first device or the second device is configured to alert the patient of the request for communication using a display, an audio signal or vibration, determine that the patient did not acknowledge the request, and automatically broadcast an emergency signal to at least one other device when the patient does not acknowledge the request.

Example 16 includes subject matter (such as a method, means for performing acts, machine readable medium including instructions that when performed by a machine cause the machine to performs acts, or an apparatus to perform). The subject matter may establish wireless communication between a first device and a second device using a communication protocol in which the first device is configured to send advertising packets and the second device is configured to discover the first device using the advertising packets and initiate the wireless communication between the first device and the second device. The subject matter may include using the first device to send advertising packets and a request to communicate with the second device, and using the second device to initiate a wireless communication session with the first device using the advertising packets.

In Example 17, the subject matter of Example 16 may optionally be configured to further include using the first device to determine a trigger for requesting communicating with the second device, and respond to the determined trigger by sending the request. The first device may request the second device to initiate communication by including embedded data in the advertising packets. The embedded data may signal a request by the first device for the second device to initiate a communication connection. The subject matter may further include using the second device to scan for advertising packets to discover the first device, recognize the embedded data in the advertising packets, and initiate the communication connection with first device in response to recognizing the embedded data.

In Example 18, the subject matter of any one or more of Examples 16-17 may optionally be configured such that the first device includes a medical device configured for use in sensing at least one health-related condition, delivering a therapy, or both sensing the at least one health-related condition and delivering the therapy.

In Example 19, the subject matter of Example 18 may optionally be configured such that the first device includes an implantable medical device.

In Example 20, the subject matter of any one or more of Examples 18-19 may optionally be configured such that may optionally be configured such that the first device includes a neuromodulator configured to deliver a neuromodulation therapy.

In Example 21, the subject matter of any one or more of Examples 16-20 may optionally be configured such that the using the first device to send advertising packets includes sending the advertising packets according to an advertising interval that specifies time between advertising events, and modifying the advertising interval in response to the determined trigger.

In Example 22, the subject matter of Example 21 may optionally be configured such that the first device is configured to determine more than one condition, and the subject matter further includes using the first device to determine at least one condition from the more than one condition, and selecting the advertising interval based on the determined at least one condition, wherein different conditions correspond to different advertising intervals.

In Example 23, the subject matter of any one or more of Examples 16-22 may optionally be configured such that the embedded data in the advertising packets is a bit to signal the request by the first device for the second device to initiate the communication connection.

In Example 24, the subject matter of any one or more of Examples 16-23 may optionally be configured such that the first device is configured for use to determine more than one condition, and the subject matter further includes the further comprising using the first device to determine at least one condition from the more than one condition, and identifying the determined at least one condition in the embedded data.

In Example 25, the subject matter of any one or more of Examples 16-24 may optionally be configured to further include sending a time stamp within the embedded data corresponding to when the determined at least one condition occurred.

In Example 26, the subject matter of any one or more of Examples 16-25 may optionally be configured to further include sending additional information relevant to the determined at least one condition, wherein the additional relevant information includes: an action by the first device, at least one measurement performed by the first device, or an indication of a condition of the first device or a person using the first device.

In Example 27, the subject matter of any one or more of Examples 16-26 may optionally be configured to further include changing operation of the first device based on the determined trigger.

In Example 28, the subject matter of Example 27 may optionally be configured such that the first device changes between a closed-loop therapy and an open loop therapy based on the determined trigger.

In Example 29, the subject matter of any one or more of Examples 27-28 may optionally be configured such that the determined trigger includes a determined medical event or a determined device event for the first device.

In Example 30, the subject matter of Example 16 may optionally be configured such that the first device is an implantable medical device configured to be implanted in a patient, the using the first device to send the request includes using the first device to alert the patient of the request for the communication, and the first device alerts the patient using a display, an audio signal, or vibration, using the first device to determine that the patient did not acknowledge the request, and automatically broadcast an emergency signal to at least one other device when the patient does not acknowledge the request.

In Example 31, the subject matter of Example 16 may optionally be configured such that the second device or a third device is configured to alert the patient of the request for the communication using a display, an audio signal, or vibration, determine that the patient did not acknowledge the request, and determine further action (e.g., automatically broadcast an emergency signal to at least one other device) when the patient does not acknowledge the request. For example, a stimulator may note an event but has no way of directly communicating with the user. For example, the stimulator may not be capable of displaying, vibrating, or producing a sound. The stimulator may send a signal to a second device (e.g., a phone) which can try to notify patient via display, vibration, or sound. The phone may determine when there is no response. Alternatively, the second device (e.g., phone) can send signal to a third device such as a BLE watch which can perform the same display, vibration, or sound to notify patient. Third device can determine that no reaction was received and inform second device.

Example 32 includes subject matter (such as a method, means for performing acts, machine readable medium including instructions that when performed by a machine cause the machine to performs acts, or an apparatus to perform). The subject matter may establish wireless communication from an implantable medical device in a patient to an external device using a generical access protocol (GAP) for a Bluetooth Low Energy (BLE) protocol where the external device is configured to operate as a central device and the implantable device is configured to operate as a peripheral device according to the GAP. The subject matter may include using the implantable medical device to send advertising packets over an advertising channel for use by the external device to discover the implantable medical device, recognize a condition, and respond to the recognized condition by requesting the external device to initiate communication with the implantable device. The implantable medical device may request the external device to initiate communication by including embedded data in the advertising packets. The embedded data may signal a request by the implantable device for the external device to initiate a communication connection. The subject matter may include using the external device to scan for advertising packets to discover the implantable medical device, recognize the embedded data in the advertising packets, and initiate the communication connection with implantable medical device in response to recognizing the embedded data.

In Example 33, the subject matter of Example 32 may optionally be configured such that the implantable medical device includes a neuromodulator or a cardiac stimulator, the external device includes a remote control, a clinician programmer, or a personal electronic device, and the recognized condition includes: abnormal sensed data; or a change in the implantable medical device. By way of example and not limitation, the personal device may include a phone, a tablet, a watch, and the like. Abnormal sensed conditions may include, but are not limited to sensed data (e.g., acceleration data indicative of a fall, eCAPs, LFPs, ERNAs, respiration (rate, volume, etc.), heart rate variability or other heart rate data such as may indicate arrhythmias such as bradycardia, tachycardia, fibrillation, or myocardial infarction, or blood pressure) outside of an expected range or sensed data that is indicative of an adverse event. Changes in the implantable medical device may include, but are not limited to changes in operation modes such changing from closed loop to open loop therapy modes, changes in lead position, changes in electrode-tissue interface (e.g., encapsulation), changes in the battery condition (e.g., need of recharge or need of replacement), and unexpected delivery of therapy.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, a known BLE protocol used to establish a communication session between a first device (e.g., GAP peripheral device) and a second device (e.g., GAP central device).

FIG. 2 illustrates, by way of example and not limitation, an embodiment of a protocol for a first device (e.g., GAP peripheral device) to notify a second device (e.g., GAP central device) of a request by the first device for the second device to establish a communication session.

FIG. 3 illustrates, by way of example and not limitation, an embodiment of a protocol for a first device (e.g., GAP peripheral device) to use advertising packets to notify a second device (e.g., GAP central device) of a request by the first device for the second device to establish a communication session.

FIG. 4 illustrates, by way of example and not limitation, an embodiment of a protocol for a first device (e.g., GAP peripheral device) to notify a second device (e.g., GAP central device) of a request by the first device for the second device to establish a communication session, wherein the request in included into the advertising packets only response to a trigger or other condition.

FIG. 5 illustrates, by way of example and not limitation, an embodiment of the first device that modifies the advertising interval in response to a detected trigger or condition.

FIG. 6 illustrates, by way of example and not limitation, examples of embedded message content that may be included into the advertising packets to request a communication connection with the second device.

FIG. 7 illustrates, by way of example, an embodiment of a neuromodulation system.

FIG. 8 illustrates, by way of example and not limitation, the neuromodulation system of FIG. 7 implemented in a spinal cord stimulation (SCS) system.

FIG. 9 illustrates, by way of example and not limitation, an embodiment of system for remotely communicating with a medical device.

FIG. 10 illustrates, by way of example and not limitation, a remote programming system for programing a therapy delivery device.

FIG. 11 illustrates, by way of example and not limitation, a healthcare monitoring system configured for use to collect healthcare-related data to be transferred to a remote system.

FIG. 12 illustrates, by way of example, an embodiment of a neuromodulation device.

FIG. 13 is a diagram illustrating a relationship between a stimulation electrode and a sensing electrode.

FIG. 14 illustrates, by way of example and not limitation, a method for providing closed-loop modulation, based on training data, using sensed electrical activity as feedback.

FIG. 15 illustrates, by way of example and not limitation, training and use of a machine-learning program.

FIG. 16 illustrates, by way of example and not limitation, a process for implementing a closed-loop therapy using a trained algorithm to control waveform parameter(s) according to signal feature feedback, including detecting anomalous feature(s) with respect to feature data used to develop the trained algorithm.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

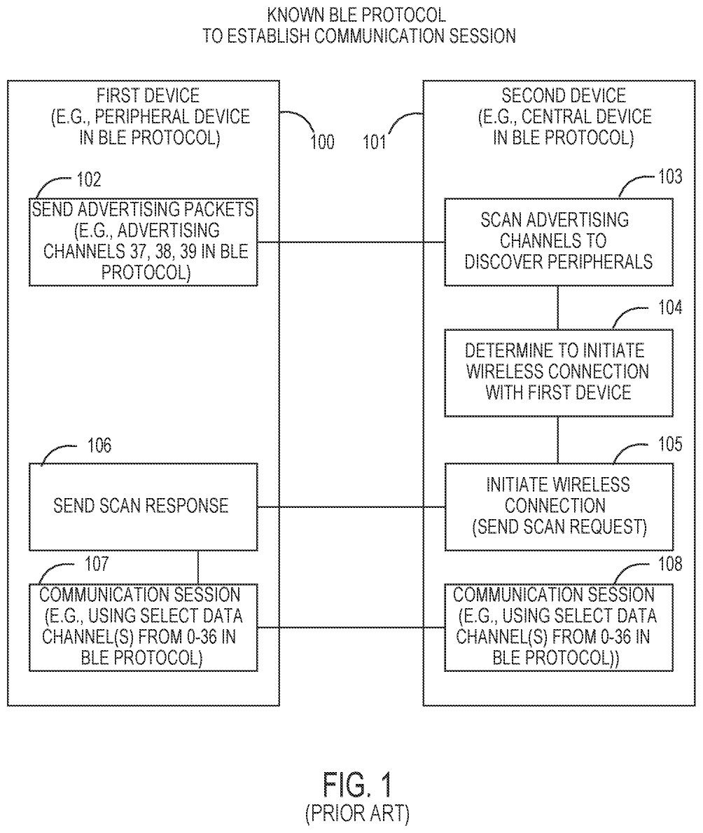

FIG. 1 illustrates, by way of example and not limitation, a known BLE protocol used to establish a communication session between a first device 100 (e.g., GAP peripheral device) and a second device 101 (e.g., GAP central device). At 102, the first device 100 advertises its presence by sending advertising packets over specified advertising channel(s). BLE peripheral devices, for example, may send BLE advertising packets out on 3 channels—37 (2402 MHz), 38 (2426 MHz), 39 (2480 MHz) for each advertising event. The advertising packets may be sent at an advertising interval which specifies the time between advertising events. The first device 100 may then wait for connection requests on the channel that was used to send the advertising packet for a specified amount of time. At 103, the second device 101 scans the specified advertising channel(s) to discover peripheral device(s) that are present and are capable of making a wireless connection for a communication session. At 104, the second device 101 determines whether to initiate a communication session with the advertising first device 100, and the second device 101 initiates the wireless connection by sending a connection request 105 to the first device 100. The connection request may include connection parameters for the new connection, where the parameters may include a connection interval indicating how often the peripheral and central devices communicate and a channel map identifying the channels on which they will communicate. The first device 100 may accept the connection by tuning into the right frequency at the right time to send a scan response 106 and establish the connection. The first device 100 and the second device 101 communicate (e.g., see communication session 107 activities performed by the first device 100 and communication session 108 activities performed by the second device 101) at the identified channels and times as long as the connection is established. BLE, for example, has specified data channels 0-36 that may be used for communication sessions.

Aspects of the present subject matter involve the first device providing a push notification to a second device where the first device is not configured to initiate a communication session. For example, various embodiments of the present subject matter implement a “push notification” routine from peripheral device to a central device. Various embodiments may implement a custom modification of the advertising packet data to relay information from the peripheral device to the central device. Thus, information such as emergency information may be sent from the peripheral device to the central device using embedded data within the broadcasted advertising packet from the peripheral device. Examples of emergencies for which it may be desirable to provide an emergency push notification may include, but are not limited to, a fall detected from IPG, a heart rate showing strange behavior, a disrupted closed-loop control, and abnormal sensed data such as abnormal evoked compound action potentials (ECAPs), local field potentials (LFPs), evoked resonant neural activity (ERNAs), and the like. The embedded data may be a bit that may function as a mailbox flag signal the central device that it should initiate a connection to peripheral device to read the underlying information. This avoids the privacy problem where patients are tracked through their resolvable private address (RPA) and advertising packet contents. The embedded data may include opcode that may identify the emergency, a timestamp for the emergency, other relevant associated with the emergency such as, by way of example and not limitation, sensing paused, or an implementation of a default stimulation program or the withdraw of stimulation.

Various embodiments may modify the advertising interval to relay the emergency information. The advertising interval may be sped up for some duration to increase the chance that the scanning external sees the embedded data. Different emergency states could affect advertising interval speed up differently. For example, a detected heart arrhythmia may correspond to a short advertising interval whereas an IPG-initiated change from closed loop to open loop may correspond to a long advertising interval.

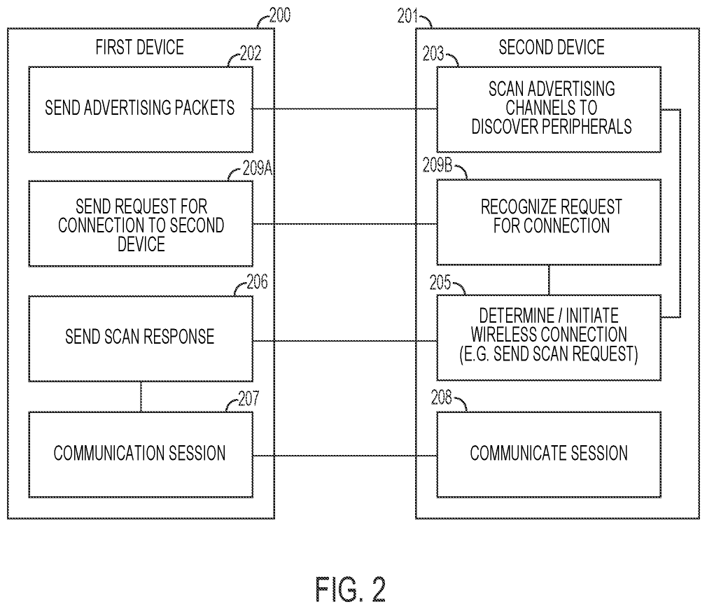

FIG. 2 illustrates, by way of example and not limitation, an embodiment of a protocol for a first device 200 (e.g., GAP peripheral device) to notify a second device 201 (e.g., GAP central device) of a request by the first device for the second device to establish a communication session. At 202, the first device 200 advertises its presence by sending advertising packets over specified advertising channel(s). At 203, the second device 201 scans the specified advertising channel(s) to discover peripheral device(s) that are present and are capable of making a wireless connection for a communication session. The first device 200 may then wait for connection requests on the channel the advertising packet was just sent out on for a specified amount of time.

Unlike the known protocol in FIG. 1, the protocol of FIG. 2 allows the first device 200 to send to the second device 201 a request for a connection 209A. The second device 201 is capable of recognizing the request for connection at 209B, and then determine/initiate the wireless connection by sending a connection request 205 to the first device 200. The first device 200 may accept the connection by tuning into the right frequency at the right time to send a scan response 206 and establish the connection. The first device 200 and the second device 201 communicate (e.g., see communication session 207 activities performed by the first device 200 and communication session 208 activities performed by the second device 201) at the identified channels and times as long as the connection is established.

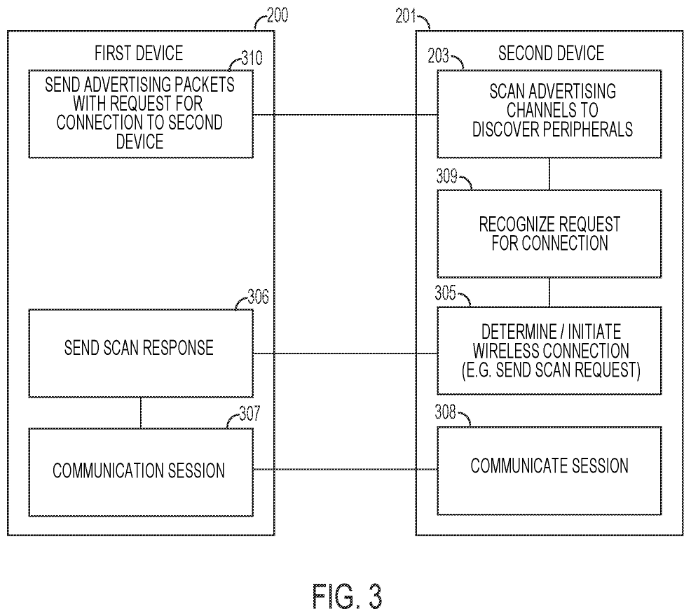

FIG. 3 illustrates, by way of example and not limitation, an embodiment of a protocol for a first device 300 (e.g., GAP peripheral device) to use advertising packets to notify a second device 301 (e.g., GAP central device) of a request by the first device for the second device to establish a communication session. In the illustrated embodiment, the request is embedded in the advertising packets. At 302, the first device 300 advertises its presence by sending advertising packets over specified advertising channel(s) where the request is included in the advertising packets, as generally illustrated at 310. At 303, the second device 301 scans the specified advertising channel(s) to discover peripheral device(s) that are present and are capable of making a wireless connection for a communication session. The second device 301 may determine to initiate the wireless connection, and initiate the wireless connection in response to the normally-broadcast advertising packets without the request. The first device 300 may then wait for connection requests on the channel used to send the advertising packet for a specified amount of time. The second device 301 is also capable of recognizing the request for connection 309 from the advertising packets, and then may determine to initiate the wireless connection, and initiate the wireless connection 305 in response to the request. The first device 300 may accept the connection by tuning into the right frequency at the right time to send a scan response 306 and establish the connection. The first device 300 and the second device 301 communicate (e.g., see communication session 307 activities performed by the first device 300 and communication session 308 activities performed by the second device 301) at the identified channels and times as long as the connection is established.

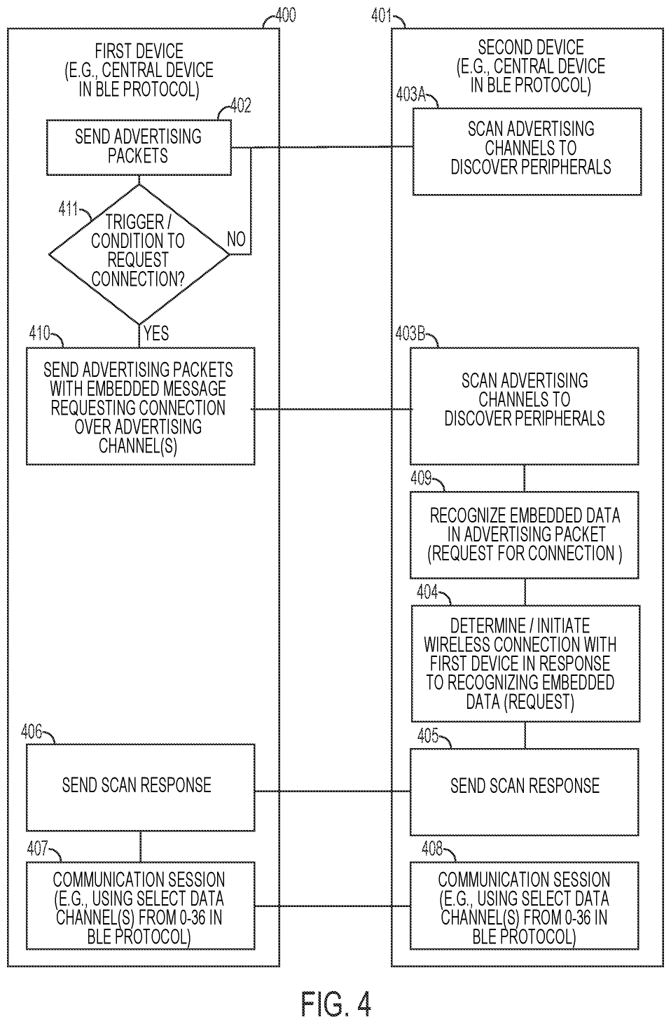

FIG. 4 illustrates, by way of example and not limitation, an embodiment of a protocol for a first device 400 (e.g., GAP peripheral device) to notify a second device 401 (e.g., GAP central device) of a request by the first device for the second device to establish a communication session, wherein the request in included into the advertising packets only response to a trigger or other condition. At 402, the first device 400 advertises its presence by sending advertising packets over specified advertising channel(s). These packets do not include a request by the first device to establish a communication connection. At 403A, the second device 401 scans the specified advertising channel(s) to discover peripheral device(s) that are present and are capable of making a wireless connection for a communication session. The second device 401 is capable of ignoring the advertisement or responding to the advertisement by sending a scan request, such as the scan request at 405. The first device 400 may then wait for connection requests on the channel the advertising packet was just sent out on for a specified amount of time. However, in the illustration, the second device 401 is ignoring the advertisement.

The first device 400 may also be configured to detect a trigger or condition at 411 which warrants a communication session with the second device. Examples of such triggers or conditions include emergency situations that warrant an emergency notification. In response to the detected trigger condition, the first device 400 may embed the request for communication within the advertising packets, and those advertising packets may be broadcast to request a connection, as illustrated at 410. The second device may scan the advertising packets to discover peripherals, and may recognize the embedded data in the advertising packets sent at 410. The second device 401 may recognize the request for connection 409 from the advertising packets, and then may determine to initiate the wireless connection 404, and initiate the wireless connection 405 in response to the request. The first device 400 may accept the connection by tuning into the right frequency at the right time to send a scan response 406 and establish the connection. The first device 400 and the second device 401 communicate (e.g., see communication session 407 activities performed by the first device 400 and communication session 408 activities performed by the second device 401) at the identified channels and times as long as the connection is established.

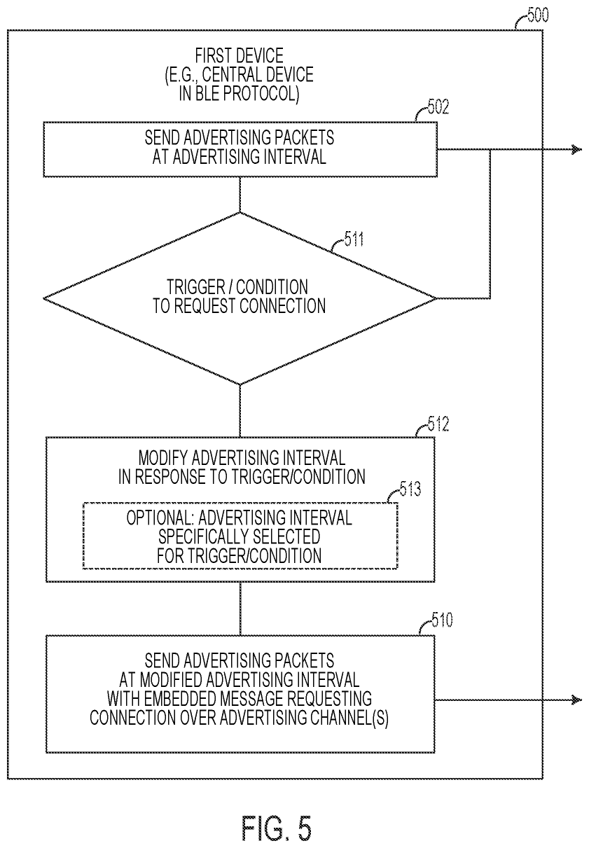

FIG. 5 illustrates, by way of example and not limitation, an embodiment of the first device that modifies the advertising interval in response to a detected trigger or condition. At 502, the first device 500 advertises its presence by sending advertising packets over specified advertising channel(s). These packets do not include a request by the first device to establish a communication connection. The second device may choose to ignore the advertisement or may choose to respond to the advertisement by sending a scan request. The first device 400 may then wait for connection requests on the channel the advertising packet was just sent out on for a specified amount of time. However, in the illustration, the second device is ignoring the advertisement. The first device 500 may detect a trigger or condition at 511 which warrants a communication session with the second device. In response to the detected trigger condition, the first device 500 may change the advertising interval, as generally illustrated at 512, and may embed the request for communication within the advertising packets as generally illustrated at 510. Those advertising packets may be broadcast at the revised advertising interval, and the second device may scan the advertising packets to discover the first device, and may recognize the embedded data in the advertising packets sent at 512. In some embodiments, as generally illustrated at 513, the revised advertising interval may be specifically selected for the specific trigger or condition that was determined at 511. Thus, if the trigger is determined to be an acute emergency, the advertising interval may be shortened, whereas other conditions may user longer advertising intervals. That is, different conditions may correspond to different advertising intervals.

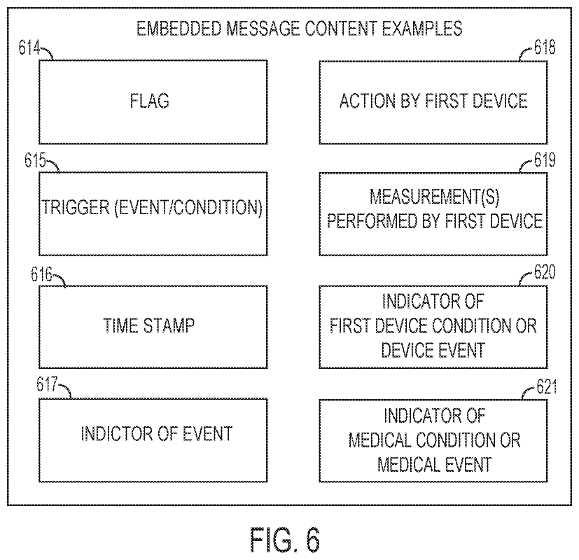

FIG. 6 illustrates, by way of example and not limitation, examples of embedded message content that may be included into the advertising packets to request a communication connection with the second device. The embedded data in the advertising packets may be a flag 614 (e.g., a bit) to signal the request by the first device for the second device to initiate the communication connection. The first device may be configured to determine more than one event or condition, the determined trigger may include at least one condition from the more than one condition, and the first device may be configured to identify the determined at least one condition in the embedded data within the advertising packets 615. The first device may be configured to send a time stamp 616 within the embedded data corresponding to when the at least one condition occurred. The first device may be configured to send additional information relevant to the determined at least one condition, and the additional relevant information may include an indicator of an event 617, an action by the first device 618, at least one measurement performed by the first device 619, or an indication of a condition of the first device 620 or an indication of a person using the first device 621. The first device may be configured to change operation based on the determined trigger. For example, the first device may change between a closed-loop therapy and an open loop therapy based on the determined trigger.

By way of example and not limitation, the IPG may detect a variety of situations for which a push notification is desirable. For example, the detected situations may be classified as an emergency using sensors such as accelerometers in the IPG, through an internal sensed signal in the brain, or some other means. These signals may indicate a potential medical emergency requiring a very quick response or may indicate an event with a high medical relevance but that is not life threatening. For example, accelerometer data may be used to identify a fall, heart rate variability, or respiration. Electrical signals sensed using electrode(s) on at least one lead may include heart rate and neural activity, including aberrant or absence of neural activity.

Falls may occur as a result of gait disturbances or other health related events, and thus are a serious concern within the movement disorder population. By way of example and not limitation, a fall may be measured by rapid acceleration followed by a sudden impact event recorded. The fall event may be further analyzed to determine if a fall event requires assistance. For example, a fall event followed by little or lack of change in sensor data for some set period may indicate that the patient has not moved or sat up or stood after the fall.

Implanted accelerometers located near the heart can detect the underlying heartbeat of the individual. This can also provide meaningful health information to the patient as an emergency event. Heart Rate Variability or arrythmias may be measured by the change in frequency patterns between successive phases of the heartbeat. A rapid heart rate may be measured by the frequency of the heartbeat can indicate stress or other medical factors. A myocardial infarction may be determined by a significant sustained change in heart rate followed by change in the pattern of cardiac signal. A loss of a heartbeat signal may indicate a cardiac arrest requiring assistance. An irregular and inconsistent cardiac rhythm may indicate an atrial fibrillation.

Respiration may cause a consistent rhythmic change in the position of the chest with inspiration and expiration, which may be detected by an accelerometer. Respiration can also provide meaningful health information to the patient as an emergency event. The respiration rate refers to the timing between inspiratory of expiratory peaks, respiratory volume refers to the amplitude between peaks of inspiration and expiration, a loss of respiration may signal a significant medical event, and high amplitude events affecting all axis occurring regularly in succession, and correlating with phase of respiration may indicate a coughing occurrence. The amplitude of the cough signal may indicate cough volume, and an average of amplitude change over time during a cough may indicate a cough flow rate.

Events that are considered to be of lesser severity may include signals related to IPG function. For example, stimulation may be off on a lead previously delivering treatment, the battery may be low or extremely low and in need of being recharged, the battery is nearing the end of life, or the IPG has entered a fallback mode. An example of a fallback mode is disabling closed loop therapy and enabling open loop therapy. The underlying issue that triggered the fallback mode may be required to be addressed before reenabling or restoring operation back from the fallback mode.

The medical device may sense or otherwise provide measurements and may monitor its own functionality for conditions or events of interest. It may be desirable to communicate these measurement(s), condition(s) or event(s) to another device or devices. For example, A user should be signaled if the battery needs to be recharged or replaced soon. By way of another example, if the stimulation is off or at 0 mA after having been on for over 24 hours (or some other set time) and this is not part of a schedule, then this may indicate a medical concern related to the function of the device that should be communicate. An emergency may be indicated after a set time in the off state when stimulation is not resumed. Sensed neural activity is expected to have certain definable characteristics. An emergency event may be determined if sensed neural activity is absent, highly repetitive (e.g., at amplitudes above noise such as a seizure), or generally outside of expected characteristics. Certain events (e.g., data anomaly, controller instability, etc.) may lead to closed-loop stimulation reverting to open-loop stimulation in a fallback mode. The external device may require notification to solve the issue or to seek help from a representative of the device manufacturer.

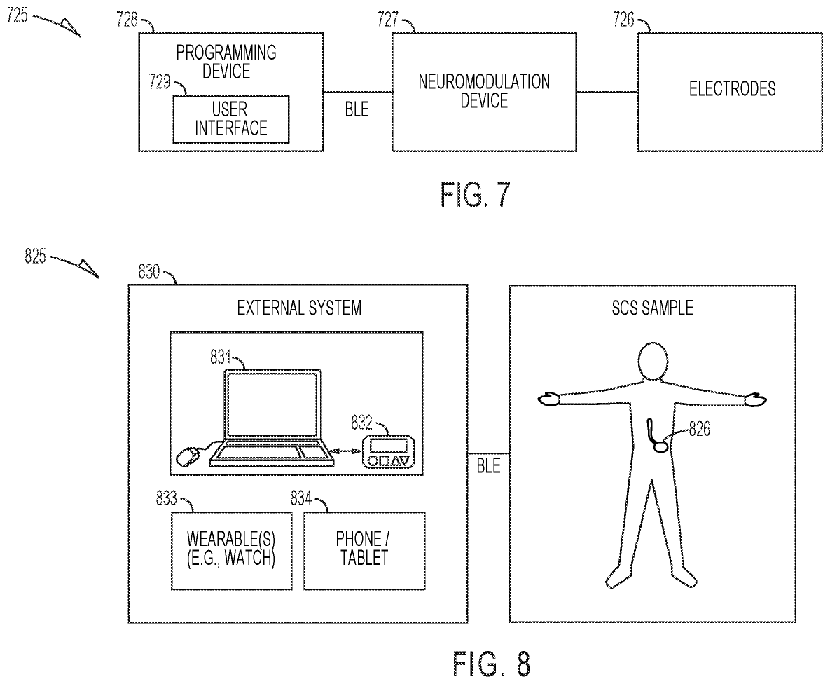

The present subject matter enables push notification from a medical device that is not configured to initiate a communication session. Examples of the medical device include a therapy-delivery device such as, but are not limited to, a neuromodulation device. Examples of a medical device include a monitor, which may but does not necessarily also delivery a therapy, as generally discussed. Examples of system that include such medical devices that may use BLE communication are generally discussed with respect to FIGS. 7-13.

After an initial activation of the device (e.g., implantation of an implantable device), the patient may be required to periodically visit the clinic in order to verify if their device is working correctly and programmed optimally. Device follow-ups may be performed by the clinicians and may be assisted by the sales representative from the device manufacturers. The present document discusses neuromodulation, also referred to as neurostimulation, as a specific example of a medical device. An implantable neuromodulation system may include an implantable neuromodulator attached to one or more implantable leads, where each lead may include one or more electrodes. The implantable neuromodulator delivers neuromodulation energy through one or more electrodes placed on or near a target site in the nervous system. An external programming device is commonly used to program the implantable neurostimulator with stimulation parameters controlling the delivery of the neurostimulation energy. Modulation parameters may comprise electrode combinations, which define the electrodes that are activated as anodes (positive), cathodes (negative), and turned off (zero), percentage of modulation energy assigned to each electrode (fractionalized electrode configurations), and electrical pulse parameters, which define the pulse amplitude (measured in milliamps or volts depending on whether the pulse generator supplies constant current or constant voltage to the electrode array), pulse width (measured in microseconds), pulse rate (measured in pulses per second), and burst rate (measured as the modulation on duration X and modulation off duration Y). The values for these parameters may be customized to a patient. The modulation parameters may be configured as a neuromodulation program capable of being implemented by the neuromodulator, and the neuromodulator may he programmed with more than one program. in order to find a program that effectively provides a therapy (e.g., pain relief) with negligible side effects, the patient or clinician may implement different programs within the neuromodulator.

FIG. 7 illustrates, by way of example, an embodiment of a neuromodulation system. The illustrated neuromodulation system 725 includes electrodes 726, a neuromodulation device 727 and a programming system such as a programming device 728, which may be a clinician programmer. The programming system may include multiple devices that may be configured to communicate with each other (e.g., remote control, clinician programmer, and mobile electronic devices such as a phone, tablet, pad and the like). The electrodes 726 are configured to be placed on or near one or more neural targets in a patient. The neuromodulation device 727 is configured to be electrically connected to electrodes 726 and deliver neuromodulation energy, such as in the form of electrical pulses, to the one or more neural targets though electrodes 726. The system may also include sensing circuitry to sense a physiological signal, which may but does not necessarily form a part of neuromodulation device 727. The delivery of the neuromodulation is controlled using a plurality of modulation parameters that may specify the electrical waveform (e.g., pulses or pulse patterns or other waveform shapes) and a selection of electrodes through which the electrical waveform is delivered. In various embodiments, at least some parameters of the plurality of modulation parameters are programmable by a user, such as a physician or other caregiver. The programming device 728 enables the user to access the user-programmable parameters, and may also provide the user with data indicative of the sensed physiological signal or feature(s) of the sensed physiological signal. In various embodiments, the programming device 728 is configured to be communicatively coupled to modulation device via a wired or wireless link. In various embodiments, the programming device 728 includes a user interface 729 such as a graphical user interface (GUI) that allows the user to set and/or adjust values of the user-programmable modulation parameters. The user interface 729 may also allow the user to view the data indicative of the sensed physiological signal or feature(s) of the sensed physiological signal and may allow the user to interact with that data. The neuromodulation device 727, the programming device 728 and other devices or system may collect data that may be used by the neuromodulation system 725. For example, the user interface 729 may be used to allow the user to answer healthcare-related questions. In various embodiments, the neuromodulation device 727 may be configured as a GAP peripheral device, the programming device may be configured as a GAP central device, and the neuromodulation device is capable of sending a push notification, e.g., via advertising packets, that requests the programming device to initiate a communication session.

FIG. 8 illustrates, by way of example and not limitation, the neuromodulation system of FIG. 7 implemented in a spinal cord stimulation (SCS) system. The illustrated neuromodulation system 825 includes an external system 830 that may include at least one programming device. The illustrated external system 830 may include a clinician programmer 831 configured for use by a clinician to communicate with and program the neuromodulator, and a remote control device 832 configured for use by the patient to communicate with and program the neuromodulator. For example, the remote control device 832 may allow the patient to turn a therapy on and off and/or may allow the patient to adjust patient-programmable parameter(s) of the plurality of modulation parameters. FIG. 8 illustrates a neuromodulation device 826 as an implantable device, although a neuromodulation device 826 may be an external device such as a wearable device. The external system 830 may include a network of computers, including computer(s) remotely located from the neuromodulation device 825 that are capable of communicating via one or more communication networks with the programmer 831 and/or the remote control device 832. The remotely located computer(s) and the neuromodulation device 826 may be configured to communicate with each other via another external device such as the programmer 831 or the remote control device 832. The remote control device 832 and/or the programmer 831 may allow a user (e.g., patient and/or clinician or rep) to answer questions as part of a data collection process. The external system 830 may include, in addition to the programmer 831 and/or remote control 832 or alternatively to the programmer 831 and/or remote control 832, a wearable 833 such as a watch, sensors or therapy-applying devices and/or a personal device 834 such as a phone or tablet. The watch may include sensor(s), such as sensor(s) for detecting activity, motion and/or posture. Other wearable sensor(s) may be configured for use to detect activity, motion and/or posture of the patient. The communication between the device 826 and the external system 830 may include a wireless communication protocol such as, but not limited to, BLE. In various embodiments, the neuromodulation device 826 may be configured as a GAP peripheral device, and one or more device(s) in the external system 830 may be configured as a GAP central device. The neuromodulation device 826 is capable of sending a push notification, e.g., via advertising packets, that requests one or more device(s) in the external system 830 to initiate a communication session.

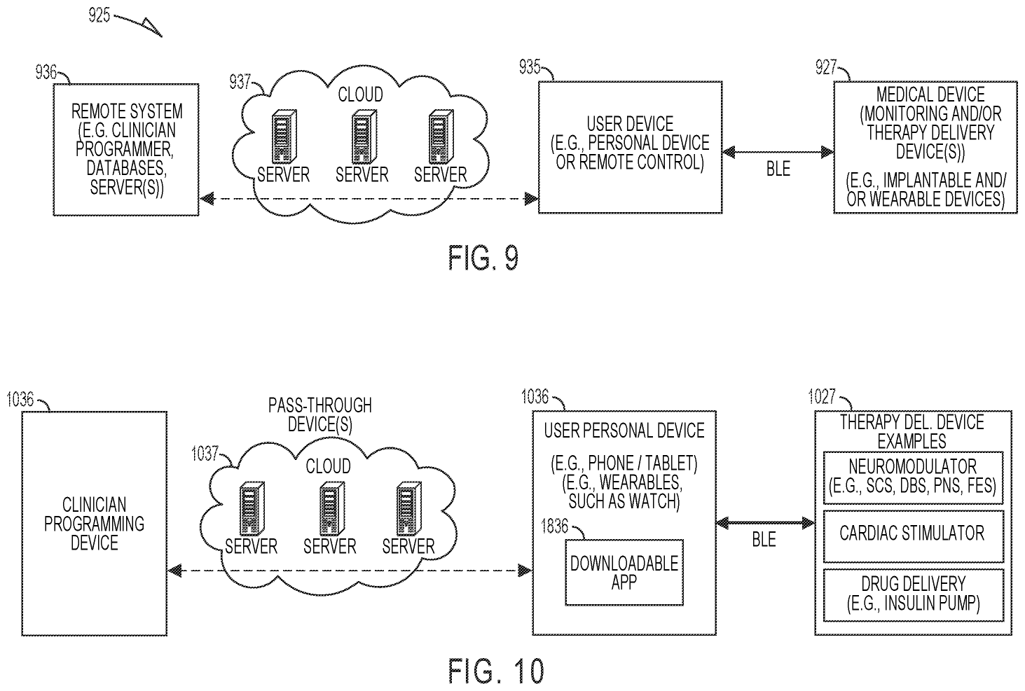

FIG. 9 illustrates, by way of example and not limitation, an embodiment of system for remotely communicating with a medical device. The system 925 may include a medical device 927, which may be an implantable device or may be a wearable device and may be configured to be a monitoring device and/or a therapy delivery device. Examples of monitoring devices include, but are not limited to, monitors that sense heart rate (or cardiac rhythm such as a Holter monitor)), blood pressure, posture, patient activity, or analytes (e.g., continuous glucose monitor). The monitor may be configured to receive various user input that may be relevant to the patient's condition, such as but not limited to diet, sleep, exercise, location and/or severity of pain, patient compliance to a therapy, and the like. The illustrated system 925 may include a user device 935, which may provide the user interface for monitor's user input. As such, the monitor functions may be performed using both the medical device 927 and the user device 935. Examples of therapy devices include electrical therapy devices such as neuromodulators and cardiac rhythm management devices, mechanical therapy devices, thermal therapy devices, and drug delivery devices. Examples of neuromodulators include, but are not limited to, spinal cord stimulators (SCS), deep brain stimulators (DBS), peripheral nerve stimulation (PNS) and function electrical stimulation (FES). Examples of cardiac rhythm management device include, but are not limited to, pacemakers and defibrillators. Examples of mechanical devices include, but are not limited to, devices configured to deliver compression to prevent deep vein thrombosis or to massage fluid from legs. Examples of drug delivery devices include, but are not limited to, insulin pumps or other infusion pumps. This disclosure discusses neuromodulation systems as a non-limiting example of a therapy device.

More particularly, FIG. 9 illustrates a system 925 that includes medical device 927, a user device 935 such as a phone, tablet or remote control configured to communicate with the medical device 927, and a remote system 936 such as a clinician programmer, database(s) such as may be used to upload monitored data from the medical device or user device, or server(s) used to download therapy programming or firmware/software updates to the medical device and/or user device. Communication between the user device 935 and the remote system 936 may pass through a number of device(s) or server(s) (e.g., “cloud” 937).

The communication between the medical device 927 and the user personal device 935 may include a wireless communication protocol such as, but not limited to, BLE. In various embodiments, the medical device 927 may be configured as a GAP peripheral device, and the user personal device 935 may be configured as a GAP central device. The medical device 927 is capable of sending a push notification, e.g., via advertising packets, that requests the user personal device 935 to initiate a communication session. Once established, the communication session may be used to communicate just with the user device 935, just with the remote system 936 using the user device 935 as a communication bridge, or with both the user device 935 and the remote system 936.

FIG. 10 illustrates, by way of example and not limitation, a remote programming system for programing a therapy delivery device 1027. The therapy-delivery device 1027 may be, by way of example, a neuromodulator, a cardiac stimulator or a drug delivery device. By way of example and not limitation, the neuromodulation device may be configured to deliver SCS, DBS, PNS or FES. The therapy-delivery device may be a neuromodulation device configured to be electrically connected to electrodes and deliver neuromodulation energy, such as in the form of electrical pulses or other waveform, to the one or more neural targets though the electrodes. The system may also include sensing circuitry to sense a biological signal, which may but does not necessarily form a part of neuromodulation device. The delivery of the neuromodulation may be controlled using a plurality of modulation parameters that may specify the electrical waveform (e.g., pulses or pulse patterns or other waveform shapes) and a selection of the electrodes through which the electrical waveform is delivered. In various embodiments, at least some parameters of the plurality of modulation parameters are programmable by a user, such as a physician or other caregiver. For example ; the parameters may comprise electrode combinations, which define the electrodes that are activated as anodes (positive), cathodes (negative), and turned off (zero), percentage of modulation energy assigned to each electrode (fractionalized electrode configurations), and electrical pulse parameters, which define the pulse amplitude, pulse width, pulse rate, and burst rate. The remote system 1036 may be configured to access and modify the user-programmable parameters, and may also provide the user with data indicative of the sensed biological signal or feature(s) of the sensed biological signal. The remote system 1036 may include a user interface such as a graphical user interface (GUI) that allows the user to set and/or adjust values of the user-programmable modulation parameters. The user interface may also allow the user to view the data indicative of the sensed biological signal or feature(s) of the sensed biological signal and may allow the user to interact with that data. The neuromodulation device, the programming device and other devices or system may collect data that may be used by the neuromodulation system. For example, a user device 1035 may have a user interface configured to enable the user to answer healthcare-related questions, such as but not limited to the efficacy of the therapy.

The therapy delivery device 1027 may provide an open-loop therapy or a closed-loop therapy. Sensing circuitry may be configured for use to detect a biological signal for use to provide feedback for the closed-loop therapy. Sensing circuitry may include various components such as an application specific integrated circuit (ASIC), hardware and/or firmware. Sensing circuitry may include software implemented using a processor to further analyze feature(s) of the biological signal. The biological signal may be a measurable signal produced by electrical, chemical or mechanical activity. Examples of electrical signals may include sensing electrical activity in the brain (e.g., EEGs), electrical activity in nerves and muscles (e.g., EMGs), cardiac activity (e.g., ECGs), and other nerve sensing including both non-evoked responses and evoked responses (e.g., evoked compound action potentials (ECAPs) or evoked resonant nerve activity (ERNA)). Examples of mechanical signals may include sounds contractions detected via flex or strain sensors. Examples of chemical signals may include detected analyte concentrations such as glucose and the like. The system may include a feature detector that is configured to detect a plurality of available features of the biological signal. At least one of the features may be used as a closed-loop sensed feature of the biological signal, which may be used by a controller to provide a closed-loop therapy. The closed-loop sensed feature may be compared to a setpoint of that feature, and the difference may be fed into a feedback control algorithm.

The user device 1035 may be a personal device of the user such as the user's smartphone, the user's tablet, or the user's wearable device such as a smart watch. The user may install a downloadable app 1038 to be executed on the user device 1035 to enable the user device to communicate with the therapy delivery device and to communicate with the remote, clinician programming device 1036 through pass-through device(s) (“cloud” 1037).

The communication between the therapy delivery device 1027 and the user personal device 1035 may include a wireless communication protocol such as, but not limited to, BLE. In various embodiments, the therapy delivery device 1027 may be configured as a GAP peripheral device, and the user personal device 1035 may be configured as a GAP central device. The therapy delivery device 1027 is capable of sending a push notification, e.g., via advertising packets, that requests the user personal device 1035 to initiate a communication session. Once established, the communication session may be used to communicate just with the user device 1035, just with the remote clinician programming device 1036 using the user device 1035 as a communication bridge, or with both the user device 1035 and the remote clinician programming device 1036.

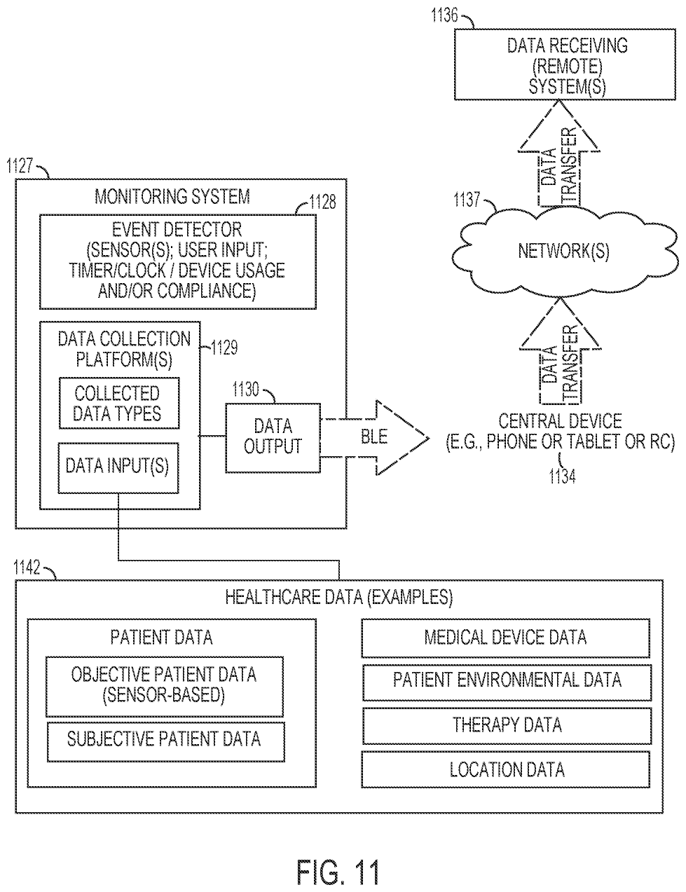

FIG. 11 illustrates, by way of example and not limitation, a healthcare monitoring system configured for use to collect healthcare-related data to be transferred to a remote system. The healthcare monitoring system 1127 may be implanted, may be wearable, or may include both implanted and wearable components. The healthcare monitoring system may include the user device, using the user interface and other features of the user device (e.g., location data) to provide healthcare-related data. The monitoring system may be configured to transfer data to a remote data receiving system 1136 for storage in a database for analysis, for example, through at least one network. The data transfer may use various network protocols to communicate and transfer data through one or more networks which may include the Internet (“cloud”) 1137 and may include various wireless networks (e.g., Wi Fi) and/or short-range wireless technology such as Bluetooth which communication uses low-power radio waves between 2.400 GHz and 2.483.5 GHz or BLE. Bluetooth communication may implement security measures. For example, “pairing” equips each device with security keys, which can be used to encrypt data, and disguise an address/identity of the device. The pairing process may authenticate devices using codes. The data may be transferred directly from at least one of the external systems and/or may be transferred directly from at least one of the medical device(s). The external system(s) may be configured to receive data from the medical device(s) and/or receive data from other healthcare-related data source(s), and then transfer the data through the network(s) to the data receiving system(s).

The illustrated monitoring system 1127 may include at least one data collection platform 1139, an event detector 1140, and a data output 1141. The data collection platform(s) 1129 may be configured to collect healthcare-related data and the data output 1130 may be configured to use BLE to transfer at least some of the collected data to a central device 1134 such as a phone or through the network(s) 1137 to a data receiving system 1136, which may include one or more server(s) or other systems remotely located from the patient. The data transfer may use various network protocols, including cryptographic protocols such as TLS, to communicate and transfer data through one or more networks which may include the Internet. The data collection platform(s) 1129 may include at least one processor configured to execute instructions stored in memory (e.g., illustrated as processor(s)/memory) to perform processes to collect and transfer data. The event detector(s) 1128 may be configured for detecting event(s), which may be used to determine when or how data is collected. The event detector 1128 may detect event(s) using sensor(s), using user input(s), using a timer or clock, using indicator(s) of device usage, patient compliance with data collection and/or therapy, or various combinations thereof. Examples of healthcare data 1142 may include patient data, medical device data, patient environmental data, therapy data, or various combinations thereof. The patient data may include objective data such as data collected from physiological sensor(s) and subjective data such as data collected from user-answered question(s) (e.g., “How do you rate your pain?”).

A monitoring system 1127 may include medical device(s), external system(s) or other healthcare related data source(s) configured for use to collect healthcare-related data for transfer to a data receiving system. One or more of the medical device(s), external system(s) or other healthcare-related data source(s) may include technology used by the system to collect data, and thus may form part of the data collection platform. The data collection platform may be on one device or may be distributed among more than one device in the system. The monitoring system may include more than one medical device configured to communicate with each other or to an external system. Examples of medical devices include implantable and wearable devices. The medical device may be configured to only collect data, to only deliver therapy, or to both collect data and deliver therapy. The medical device may include sensor(s) configured for use to collect patient data (e.g., objective patient data). The medical device may be configured to collect and provide medical device data such as device model, configuration, settings, and the like. Thus, the medical device may be configured to collect patient data, medical device data, environmental data, and therapy data such as stimulation settings. Examples of external system(s) include remote controls, programmers, and personal devices such as phones, tablets, smart watches, personal computers, and the like. The external system(s) may include at least one user interface configured for use to receive user input, which may include user answers to questions. The user answers received via the user interface(s) may include subjective patient data (e.g., “How do you rate your pain?” or “Where do you feel pain?”) or objective patient data (e.g., “What is your heart rate?”, “What is your blood pressure?”, or “When did you fall asleep and wake up?”). The external system may be configured to collect medical device data from the medical device. Other healthcare-related data source(s) may include patient data received via a provider's server that stores patient health records. For example, patients may use a patient portal to access their health records such as test results, doctor notes, prescriptions, and the like. Other healthcare-related data sources may include various apps on a patient's phone or other device, or the data on a server accessed by those apps. By way of example and not limitation, this type of data may include heart rate, blood pressure, weight, respiration activity, muscle activity, analyte measurements (e.g., glucose measurements from a continuous glucose monitor), and the like. An app on a phone or patient's device may include or may be configured to access environmental data such as weather data and air quality information or location elevation data such as may be determined using cellular networks and/or a global positioning system (GPS). Weather data may include, but is not limited to, barometric pressure, temperature, sunny or cloud cover, wind speed, and the like.

The communication between the monitoring system 1127 and the central device 1134 (e.g., phone or tablet) may include a wireless communication protocol such as, but not limited to, BLE. In various embodiments, the monitoring system 1127 may be configured as a GAP peripheral device. The monitoring system is capable of sending a push notification, e.g., via advertising packets, that requests the central device 1134 to initiate a communication session. Once established, the communication session may be used to communicate just with the central device 1134, just with the remote system 1136 using the central device 1134 as a communication bridge, or with both the central device 1134 and the remote system 1136.

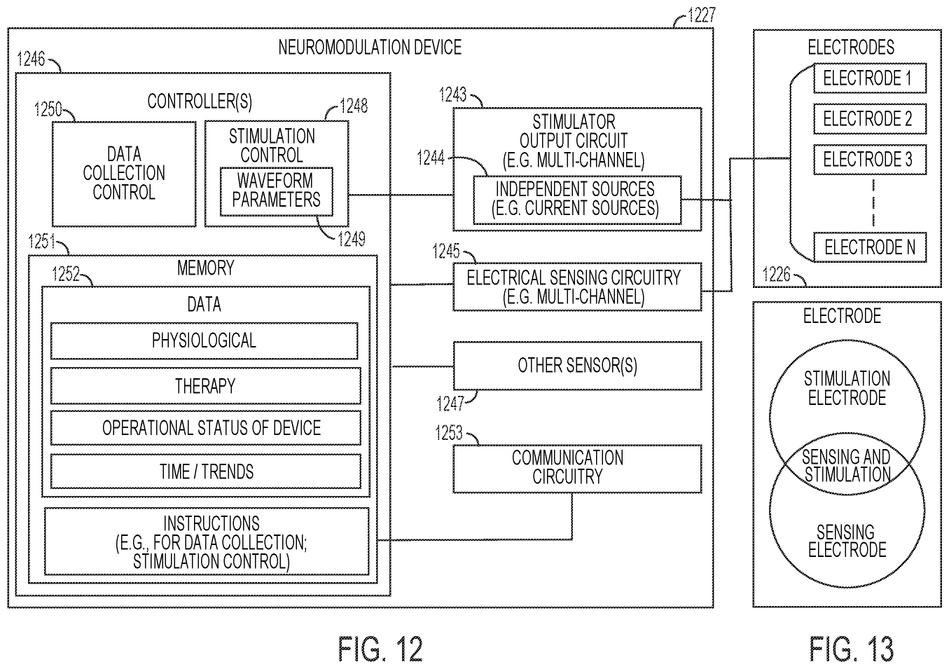

FIG. 12 illustrates, by way of example, an embodiment of a neuromodulation device. The neuromodulation device 1227 may be configured to be connected to electrode(s) 1226, illustrated as N electrodes. Any one or more of the electrodes 1226 may be configured for use to deliver modulation energy, sense electrical activity, or both deliver modulation energy and sense electrical activity (see FIG. 13). The neuromodulation device 1227 may include a stimulator output circuit 1243 configured to deliver modulation energy to electrode(s). The stimulator output circuit 1243 may be configured with multiple (e.g., two or more) channels for delivering modulation energy, where each channel may be independently controlled with respect to other channel(s). For example, the stimulator output circuit 1243 may have independent sources 1244 such as independent current sources or independent voltage sources. The neuromodulation device 1227 may include electrical sensing circuitry 1245 configured to receive sensed electrical energy from the electrode(s), such as may be used to sense electrical activity in neural tissue or muscle tissue. The sensing circuitry may be configured to process signals in multiple (e.g., two or more) channels. By way of example and not limitation, the electrical sensing circuitry 1245 may be configured to amplify and filter the signal(s) in the channel(s). The controller 1246 may be configured to detect one or more features in the sensed signals. Examples of features that may be detected include peaks (e.g., minimum and/or maximum peaks including local peaks/inflections), range between minimum/maximum peaks, local minima and/or local maxima, area under the curve (AUC), curve length between points in the curve, oscillation frequency, rate of decay after a peak, a difference between features, and a feature change with respect to a baseline. Detected feature(s) may be fed into a control algorithm, which may use relationship(s) between the feature(s) and waveform parameter(s) to determine feedback for closed-loop control of the therapy. Some embodiments of the modulation device 517 may include or be configured to receive data from other sensor(s) 1247. The other sensor(s) 1247 may include physiological sensor(s), environmental sensor(s), or proximity sensor(s). The controller 1246 operably connected to the stimulator output circuit 1243 and the sensing circuitry 1245, 1246. The controller 1246 may include a stimulation control 1248 configured for controlling the stimulator output circuit 1243. For example, the stimulation control 1248 may include start/stop information for the stimulation and/or may include relative timing information between stimulation channels. The stimulation control 1248 may include waveform parameters 1249 that control the waveform characteristics of the waveform produced by the stimulation output circuit 1243. The waveform parameters 1249 may include, by way of example and not limitation, amplitude, frequency, and pulse width parameters. The waveform parameters 1249 may include, by way of example and not limitation, regular patterns such as patterns regularly repeat with same pulse-to-pulse interval and/or irregular patterns of pulses such as patterns with variable pulse-to-pulse intervals. The waveform parameters may, but do not necessary, define more than one waveform shape (e.g., including a shape other than square pulses with different widths or amplitudes). The stimulation control 1248 may be configured to change waveform parameter(s) (e.g., one or more waveform parameters) in response to user input and/or automatically in response to feedback.

The controller 1246 may include a data collection control 1250 configured for use to collect healthcare related data. The controller 1246 may include a memory 1251 with instructions 1252 for use to control the data collection using the data collection control 1250 and control the stimulation via the stimulation control 1248. The memory 1251 may also include storage for storing different types of collected healthcare-related data 1252, such as physiological data, therapy data, data regarding the operational status of the device, and times/trends for data. Examples of physiological data may include, by way of example and not limitation, heart rate, heart rate variability, oxygen saturation, activity, posture, steps, gait, temperature, evoked compound action potentials (ECAPS), electromyograms (EMGs), electroencephalograms (EEGs), weight, blood pressure, and the like. Examples of therapy data may include, by way of example and not limitation, stimulations settings such as amplitude, pulse width, pulse frequency period, duration of burst of pulses, active electrodes, electrode fractionalization controlling the distribution of energy (e.g., current) to active electrodes, waveforms, pulse patterns including various complex patterns, and the like. Examples of data regarding the operational status of the device may include, by way of example and not limitation, electrode-tissue impedance, fault conditions, battery information such as battery health, battery life, voltage, charge state, charging history if rechargeable, MM status, Bluetooth connection logs, connection with Clinician Programmer, hours of operation/duration of implant, and the like. Other device information may include device model and lead model. Examples of time or trend data may include changes (e.g., increases and/or decreases) in activity, pain, function and sleep. The neuromodulation device may include communication circuitry 1253 configured for use to communicate with other device(s) such as a programmer, remote control, phone, tablet and the like. The healthcare-related data may be transferred out from the neuromodulation device for transfer to a data receiving system.

FIG. 13 is a diagram illustrating a relationship between a stimulation electrode and a sensing electrode. The stimulation electrode is configured for use in delivering modulation energy, and the sensing electrode is configured for use in sensing electrical activity. As illustrated, the stimulation electrode may also be used in sensing electrical activity, and the sensing electrode may also be used in delivering modulation energy. Thus, the term “stimulation electrode” does not necessary exclude the electrode from also being used to sense electrical activity; and the term “sensing electrode” does not necessarily exclude the electrode from also being used to deliver modulation energy.

From the previous discussion of FIGS. 7-13, those of ordinary skill in the art, who has read and comprehended this disclosure, will understand that the medical device, which may be configured as a GAP peripheral device, is capable of generating a significant amount of data related to the sensors, patient condition, medical device condition, and therapy status. This data may be used to identify, using previously identified criteria, a condition or trigger that warrants the medical device to send a push notification, e.g., using advertising packets, that requests a communication session with a central device.

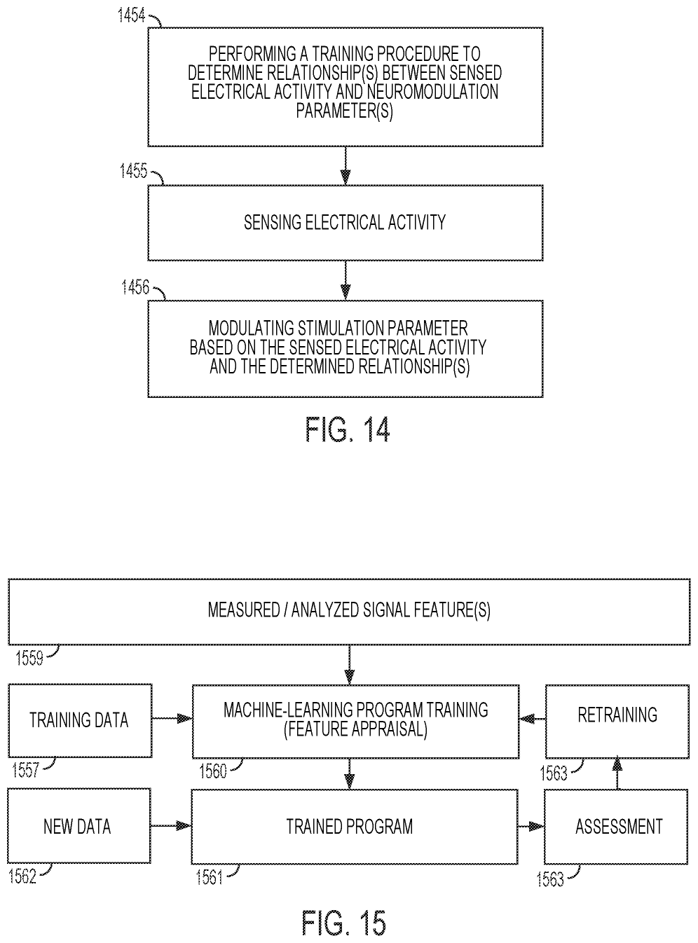

By way of example, it is noted that anomalous data may trigger a push notification. For example, machine learning may be used to develop an algorithm using training data. A therapy may use the developed algorithm to control the therapy. However, various embodiments monitor the data for anomalies to prevent the therapy from being delivered using anomalous data. FIGS. 14-16 provide additional details regarding data anomalies.

FIG. 14 illustrates, by way of example and not limitation, a method for providing closed-loop modulation, based on training data, using sensed electrical activity as feedback. The training data may be used by a machine learning algorithm to determine relationship(s) between the sensed electrical activity (e.g., extracted feature(s) of an electrical signal) and the parameter(s) of the neuromodulation. The method may include, at 1454, performing a training procedure to determine a relationship between sensed electrical activity and neuromodulation parameters. Examples of sensed electrical activity includes neural activity or muscle activity. Examples include local field potentials, evoked compound action potentials (ECAPs), or evoked resonant neural activity (ERNA). For example, a training procedure may be performed by delivering the neurostimulation energy at one or more neurostimulation intensity levels to a neural target of the patient for evaluation. Feature(s) may be extracted from the sensed signal, and a relationship may be determined between the extracted feature(s) and the neuromodulation parameter(s) using mathematical or statistical modeling of the extracted feature(s). At 1455, a physiological signal, such as electrical activity, is sensed, and stimulation parameters may be modulated according to the sensed electrical activity and the determined relationship, as illustrated at 1456. Various stimulation parameters can be modulated, including but not limited to: current amplitude, frequency, pulse width, duty cycle, stimulation fractionalization, waveform shapes, waveform patterns (e.g., regular and/or irregular patterns of pulses or trains of pulses), stimulation on/off times, and combinations thereof.

FIG. 15 illustrates, by way of example and not limitation, training and use of a machine-learning program. In some example embodiments, machine-learning programs (MLPs), also referred to as machine-learning algorithms or tools, are utilized to perform operations associated with machine learning tasks, such as identifying relationship(s) between detected feature(s) in a sensed biological signal and waveform parameter(s) used to control the neuromodulation. Thus, machine learning may be used to determine the relationships between the extracted features and the stimulation therapy.

Machine learning is a field of study that gives computers the ability to learn without being explicitly programmed. Machine learning explores the study and construction of algorithms that may learn from existing data (e.g., “training data” or “learning data”) and make predictions about new data. Such machine-learning tools may build a model from example training data 1557 in order to make data-driven predictions or decisions expressed as outputs or 1558. The machine-learning algorithms may use the training 1557 to find correlations among identified features 1559 that affect the outcome.

The machine-learning algorithms use features 1559 for analyzing the data to generate assessments 1558. A feature is an individual measurable property of the observed phenomenon. In the context of a biological signal, some examples of features may include, but are not limited to, peak(s) such as a minimum peak, a maximum peak as well as local minimum and maximum peaks, a range between peaks, a difference in values for features, a feature change with respect to a baseline, an area under a curve, a curve length, an oscillation frequency, and a rate of decay for peak amplitude. Inflection points in the signal may also be an observable feature of the signal, as an inflection point is a point where the signal changes concavity (e.g., from concave up to concave down, or vice versa), and may be identified by determining where the second derivative of the signal is zero. Detected feature(s) may be partially defined by time (e.g., length of curve over a time duration, area under a curve over a time duration, maximum or minimum peak within a time duration, etc.). The features may include time domain features, frequency domain features, or wavelet domain features.

The machine-learning algorithms use the training data 1557 to find correlations among the identified features 1559 that affect the outcome or assessment 1558. With the training data 1557 and the identified features 1559, the machine-learning tool is trained at operation 1560. The machine-learning tool appraises the value of the features 1559 as they correlate to the training data 1557. The result of the training is the trained machine-learning program 1561. Various machine learning techniques may be used to train models to make predictions based on data fed into the models. During a learning phase, the models are developed against a training dataset of inputs to optimize the models to correctly predict the output for a given input. A training data set may be defined for desired functionality of the closed-loop algorithm and closed loop parameters may be defined for desired functionality of the closed-loop algorithm. Generally, the learning phase may be supervised, semi-supervised, or unsupervised; indicating a decreasing level to which the “correct” outputs are provided in correspondence to the training inputs. In a supervised learning phase, all of the outputs are provided to the model and the model is directed to develop a general rule or algorithm that maps the input to the output. In contrast, in an unsupervised learning phase, the desired output is not provided for the inputs so that the model may develop its own rules to discover relationships within the training dataset. In a semi-supervised learning phase, an incompletely labeled training set is provided, with some of the outputs known and some unknown for the training dataset.

Models may be run against a training dataset for several epochs (e.g., iterations), in which the training dataset is repeatedly fed into the model to refine its results. For example, in a supervised learning phase, a model is developed to predict the output for a given set of inputs, and is evaluated over several epochs to more reliably provide the output that is specified as corresponding to the given input for the greatest number of inputs for the training dataset. In another example, for an unsupervised learning phase, a model is developed to cluster the dataset into n groups, and is evaluated over several epochs as to how consistently it places a given input into a given group and how reliably it produces the n desired clusters across each epoch.

Once an epoch is run, the models are evaluated and the values of their variables are adjusted to attempt to better refine the model in an iterative fashion. In various aspects, the evaluations are biased against false negatives, biased against false positives, or evenly biased with respect to the overall accuracy of the model. The values may be adjusted in several ways depending on the machine learning technique used. For example, in a genetic or evolutionary algorithm, the values for the models that are most successful in predicting the desired outputs are used to develop values for models to use during the subsequent epoch, which may include random variation/mutation to provide additional data points. One of ordinary skill in the art will be familiar with several other machine learning algorithms that may be applied with the present disclosure, including linear regression, neural networks, and the like.

New data 1562 is provided as an input to the trained machine-learning program 1561, and the trained machine-learning program 1561 generates the assessment 1558 as output. The outputted assessment 1558 may be out of an expected range (e.g., anomalous), indicating that remedial action such as retraining 1563 of the machine learning algorithm(s) is warranted. The system also may be configured to determine that the new data 1562 includes anomalous data with respect to the training data 1557 that was used to train the machine-learning program. The detection of new data that is anomalous may trigger remedial action(s) such as, if it is determined that the previously used training data is outdated, retraining 855 the machine learning program using updated training data. By way of example and not limitation, the remedial action may be initially triggered using a push notification of the anomalous data, which alerts a central device of the need to establish a communication session, which can then be used to communicate that remedial action should be taken to retrain or otherwise address the anomalous data, or to communicate that the device is changing operational modes to appropriately accommodate that the data is determined to be anomalous.

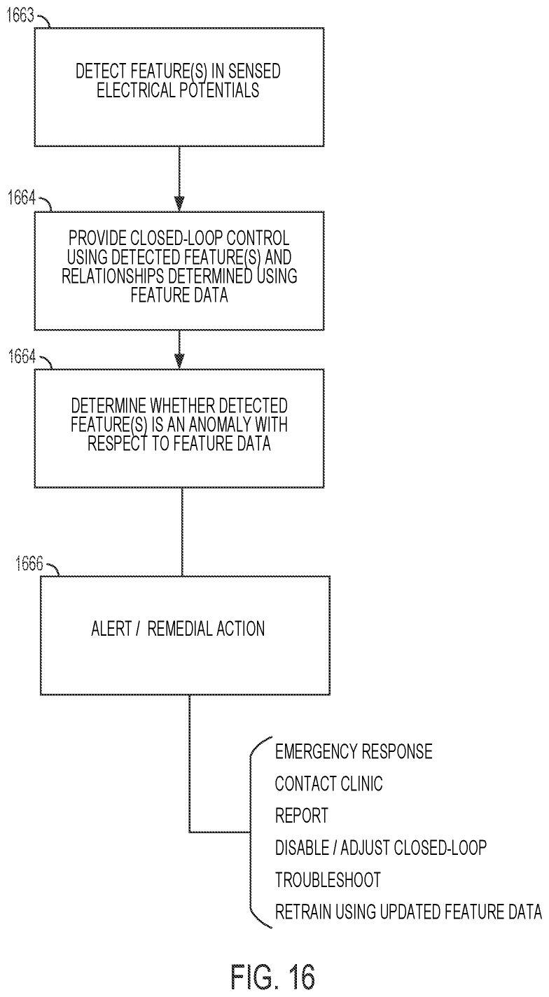

FIG. 16 illustrates, by way of example and not limitation, a process for implementing a closed-loop therapy using a trained algorithm to control waveform parameter(s) according to signal feature feedback, including detecting anomalous feature(s) with respect to feature data used to develop the trained algorithm. The illustrated process for implementing the closed-loop therapy includes detecting feature(s) within sensed electrical potentials, at 1663, and providing closed-loop control using the detected feature(s) and relationship(s) determined using feature data (e.g., trained algorithm developed or trained based on the feature data) 1664. The illustrated process may further include determining whether detected feature(s) is (are) an anomaly with respect to the feature data used to determine the relationship(s) 1665. As illustrated at 1666, remedial action may be performed upon determining that detected feature(s) is (are) anomalous with respect to the feature data used to develop the trained algorithm). An initial step for a remedial action may be for the peripheral device to send a push notification to a central device indicating a need for the central device to establish a communication session with the peripheral device. This communication session may be used to enable the remedial action to be performed and/or communicated. The remedial action for a detected anomaly may be an emergency response, initiating communication to the clinic, or providing a report. By way of example and not limitation, the remedial action for a detected anomaly may include disabling a closed-loop control of the therapy. The therapy may be stopped completely, or may implement an open-loop therapy based on previously-determined waveform parameters. The remedial action for a detected anomaly may include adjusting the closed-loop therapy. The closed-loop therapy may be adjusted by adjusting the parameters and/or adjusting or replacing the algorithm itself. For example, the closed-loop algorithm may be adjusted by adjusting parameter value thresholds or functions (e.g., transfer function) implemented by the closed-loop algorithm to provide closed-loop control of the therapy. The remedial action for a detected anomaly may include presenting a troubleshooting question(s) and/or presenting reports to a patient or other user (e.g., via a remote control or other patient device (e.g., smartphone, a programmer, or a local or remote computer). The remedial action for a detected anomaly may include initiating a retraining of the trained algorithm using updated feature data to update the relationship(s) between detected feature(s) and waveform parameter(s). The remedial action may include manual and/or automatic activities. In some embodiments, the remedial action includes reconfiguring a sensing configuration. For example, signal processing such filtering, averaging, and/or sensing electrode selection may be changed as part of the remedial action.

The anomaly detection may be performed in real-time or in near real-time, or may be intermittently performed as an audit of stored feature data. For example, the anomaly detector may look for anomalies every epoch, such as a stimulation epoch between stimulation pulses or a control epoch between times when the programmed control algorithm executes and updates stimulation. The anomaly detector timing may use “instances” of detected features, which indicates times when a set of one or more feature(s) are extracted from a sensed signal. The instances of detected features may correspond to a control epoch, reflecting a period of time when the programmed control algorithm executes, analyzes sensor activity, and updates stimulation. However, a single control epoch may include one or more instances of detecting/extracting a set of one or more feature(s) in the sensed signal(s). The programmed control algorithm may be configured to use more than one feature detection instance to control the therapy. The anomaly detection may be configured to audit stored feature data (or stored signals) every time period (e.g., one the order of hours, days, or weeks). The time period may space across a plurality of instances of detected features or control epochs. The time period may be adjusted by the system or a user of the system.

In addition or alternative to communicating a push notification from the IPG using advertising packets, the IPG may attempt to use a display, tone, vibration, or other means of recognition to inform the patient that they have a serious alert and must acknowledge. More serious emergency situations may have different methods of communication to indicate seriousness (i.e., loud ringing vs. small buzz). The acknowledgement may be indicated by tapping the IPG twice or through interaction with an external device in communication with the IPG. If acknowledgement is made, the nature of the emergency can be shown on the RC or some other external device.

When it is a medical emergency, the device may ask or recommend that the patient immediately contact their hospital or emergency services. The device can then facilitate this communication. If the signal is an emergency signal and the patient does not acknowledge the alert, then the emergency signal may be automatically broadcast to additional devices which could include but are not limited to an emergency service, a hospital or physician, a representative of the device manufacturer, a caregiver, and/or another emergency contact. The device may detect anomalous data. For example, if measurements consistently indicate potential emergency events but they are not sustained, the device may send a notification to a connected patient external device or to the physician suggesting medical follow up.

Devices may include a broadcaster that may be a non-connectable advertiser, observer scanner that does not initiate connections, a peripheral connectable advertiser that does not initiate connections, a central scanner that initiates connections. An IPG may be configured to function as a multi role device (i.e., a central device for limited amount of time when emergency occurs). An external device may have a second advertising channel looking for this connection.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encrypted with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method for establishing wireless communication between a first device and a second device using a communication protocol in which the first device is configured to send advertising packets and the second device is configured to discover the first device using the advertising packets and initiate the wireless communication between the first device and the second device, the method comprising:
using the first device to send advertising packets and a request to communicate with the second device; and
using the second device to initiate a wireless communication session with the first device using the advertising packets.

2. The method of claim 1, further comprising:
using the first device to determine a trigger for requesting communicating with the second device, and respond to the determined trigger by sending the request, wherein the first device requests the second device to initiate communication by including embedded data in the advertising packets, wherein the embedded data signals a request by the first device for the second device to initiate a communication connection; and
using the second device to scan for advertising packets to discover the first device, recognize the embedded data in the advertising packets, and initiate the communication connection with first device in response to recognizing the embedded data.

3. The method of claim 1, wherein the first device includes a medical device configured for use in sensing at least one health-related condition, delivering a therapy, or both sensing the at least one health-related condition and delivering the therapy.

4. The method of claim 3, wherein the first device includes an implantable medical device.

5. The method of claim 3, wherein the first device includes a neuromodulator configured to deliver a neuromodulation therapy.

6. The method of claim 1, wherein the using the first device to send advertising packets includes sending the advertising packets according to an advertising interval that specifies time between advertising events, and modifying the advertising interval in response to the determined trigger.

7. The method of claim 6, wherein the first device is configured to determine more than one condition, the method further comprising using the first device to determine at least one condition from the more than one condition, and selecting the advertising interval based on the determined at least one condition, wherein different conditions correspond to different advertising intervals.

8. The method of claim 1, wherein the embedded data in the advertising packets is a bit to signal the request by the first device for the second device to initiate the communication connection.

9. The method of claim 1, wherein the first device is configured for use to determine more than one condition, the further comprising using the first device to determine at least one condition from the more than one condition, and identifying the determined at least one condition in the embedded data.

10. The method of claim 9, further comprising sending a time stamp within the embedded data corresponding to when the determined at least one condition occurred.

11. The method of claim 10, further comprising sending additional information relevant to the determined at least one condition, wherein the additional relevant information includes: an action by the first device, at least one measurement performed by the first device, or an indication of a condition of the first device or a person using the first device.

12. The method of claim 10, further comprising changing operation of the first device based on the determined trigger.

13. The method of claim 12, wherein the first device changes between a closed-loop therapy and an open loop therapy based on the determined trigger.

14. The method of claim 12, wherein the determined trigger includes a determined medical event or a determined device event for the first device.

15. The method of claim 1, wherein:
the first device is an implantable medical device configured to be implanted in a patient,
the using the first device to send the request includes using the first device to alert the patient of the request for the communication,
the first device alerts the patient using a display, an audio signal, or vibration,
using the first device to determine that the patient did not acknowledge the request, and
automatically broadcast an emergency signal to at least one other device when the patient does not acknowledge the request.

16. The method of claim 1, wherein the second device or a third device is configured to alert the patient of the request for the communication using a display, an audio signal, or vibration, determine that the patient did not acknowledge the request, and automatically broadcast an emergency signal to at least one other device when the patient does not acknowledge the request.

17. A method for establishing wireless communication from an implantable medical device in a patient to an external device using a generical access protocol (GAP) for a Bluetooth Low Energy (BLE) protocol where the external device is configured to operate as a central device and the implantable device is configured to operate as a peripheral device according to the GAP, the method comprising:
using the implantable medical device to:
send advertising packets over an advertising channel for use by the external device to discover the implantable medical device;
recognize a condition; and
respond to the recognized condition by requesting the external device to initiate communication with the implantable device, wherein the implantable medical device requests the external device to initiate communication by including embedded data in the advertising packets, wherein the embedded data signals a request by the implantable device for the external device to initiate a communication connection;
using the external device to:
scan for advertising packets to discover the implantable medical device;
recognize the embedded data in the advertising packets; and
initiate the communication connection with implantable medical device in response to recognizing the embedded data.

18. The method of claim 17, wherein:
the implantable medical device includes a neuromodulator or a cardiac stimulator;
the external device includes a remote control, a clinician programmer, or a personal electronic device; and
the recognized condition includes: abnormal sensed data; or a change in the implantable medical device.

19. A system, comprising:
a first device and a second device,
wherein the first device is configured to send advertising packets and send a request to communicate with the second device, and
wherein the second device is configured to discover the first device and initiate a wireless communication session with the first device using the advertising packets.

20. The system of claim 19, wherein:
the first device is configured to determine a trigger for requesting communicating with the second device, and respond to the determined trigger by sending the request, wherein the first device requests the second device to initiate communication by including embedded data in the advertising packets, and wherein the embedded data signals a request by the first device for the second device to initiate a communication connection; and
the second device is configured to scan for advertising packets to discover the first device, recognize the embedded data in the advertising packets, and initiate the communication connection with first device in response to recognizing the embedded data.
